(19) United States
(12) Patent Application Publication
(10) Pub. No.: US 2018/0168730 A1
(43) Pub. Date: Jun. 21, 2018

(54) SYSTEM AND METHOD FOR MEDICAL PROCEDURE PLANNING

(71) Applicant: ZAXIS LABS, Chicago, IL (US)

(72) Inventor: Nuha NAZY, Bushkill, PA (US)

(73) Assignee: ZAXIS LABS, Chicago, IL (US)

(21) Appl. No.: 15/560,742

(22) PCT Filed: Mar. 25, 2016
(86) PCT No.: PCT/US16/24294

§ 371 Date: Sep. 22, 2017

Related U.S. Application Data
(60) Provisional application No. 62/138,083, filed on Mar. 25, 2015.

(51) Int. Cl. A61B 34/10; G05B 19/4099; G16H 20/30; G16H 30/40; B33Y 50/00; B33Y 80/00; G06T 15/10; G06T 19/00

(52) U.S. Cl. CPC A61B 34/10; G05B 19/4099; G16H 20/30; G16H 30/40; B33Y 50/00; B33Y 80/00; G06T 2210/41; G06T 19/003; A61B 2034/105; A61B 2034/108; G05B 2219/35134; G05B 2219/49007; G06T 15/10

(57) ABSTRACT

A system and method of interactively communicating and displaying patient-specific information is provided. The method includes acquiring two-dimensional or three-dimensional computer images of a patient-specific body part and using a computer to generate an interactive three-dimensional computer model based off of the acquired computer images of the patient-specific body part. Further, the method includes generating a physical three-dimensional model based off of the computer model, and incorporating one or more indicators into the physical three-dimensional model. Each indicator is in communication with the computer. Furthermore, the method includes interacting with the computer model to select an attribute of the body part, and indicating on the physical three-dimensional model the selected attribute with the indicators.

### CROSS REFERENCE TO RELATED APPLICATIONS

The present application claims the benefit of U.S. Provisional Patent Application No. 62/138,083 filed Mar. 25, 2015 entitled “METHOD OF 3D PRINTING INTERNAL STRUCTURES” the entire disclosure of which is hereby incorporated by reference herein.

### BACKGROUND OF THE INVENTION

The present invention concerns in general the optimization of procedures in radiological diagnostics. The present invention more particularly concerns an intelligent and thus adaptive data acquisition or image processing in order to achieve an improvement with regard to interface design, training and documentation in radiological image-processing evaluation of medical findings.

Human anatomy is cognitively difficult to master using only two-dimensional (2D) medical imaging tools. Three-dimensional (3D) visualization and 3D printing in surgical practice represents an aspect of personalized medicine and exposes tremendous potential to the field of surgical preparation and medical training.

Conventional training tools currently available are often focused on specific skills development (e.g., laparoscopic training using blocks), which are rudimentary or rote. Cadaver labs expose the students only to the conditions the cadaver presents. There is very limited opportunity for a surgical student to practice e.g., a pancreatic whipple procedure in advance of training on a living patient.

The present invention addresses the foregoing deficiencies in the prior art.

### BRIEF SUMMARY OF THE INVENTION

In accordance with the present invention, the problems and limitations of conventional imaging technologies and methods for surgical preparation is solved by engendering a mixed reality simulation system that incorporates various technologies into a process to consistently achieve a desired result that simulates the surgical environment and produces tangible products for planning and preparation. In this way, the system can be optimized to achieve the best results for the intended process and application.

In accordance with a preferred embodiment, the present invention provides a method for interactively communicating and displaying patient-specific information including acquiring two-dimensional or three-dimensional images of a patient-specific body part, using a computer, generating an interactive three-dimensional computer model based off of the acquired images of the patient-specific body part, generating a physical three-dimensional object based off of the computer model, incorporating one or more indicators into the physical three-dimensional object that are each in communication with the computer, interacting with the computer model to select an attribute of the body part, and indicating on the physical three-dimensional object the selected attribute with the indicators.

The method can also include the steps of receiving a selection of a feature of the patient-specific body part for providing interactive responsiveness, and positioning the indicator in the cavity, the indicator operable to respond to a signal from the computer and provide a physically tangible response associated with the three-dimensional object. The step of generating a physical three-dimensional object comprises printing a plurality of pieces that assemble to form the physical three-dimensional object and the assembled physical three-dimensional object defines a cavity suitable to receive the indicator, and the step of incorporating the indicators into the physical three-dimensional object is conducted during the step of generating the physical three-dimensional computer model. The indicators can be visual, audio or vibratory indicators. The step of interacting with the computer model, comprises conducting a fly-through of the interactive three-dimensional computer model. The step of generating the physical three-dimensional object includes printing the physical three-dimensional object.

In accordance with another preferred embodiment, the present invention provides a method of developing a medical treatment plan comprising, acquiring two-dimensional or three-dimensional computer images of a patient-specific body part, identifying a target area of the patient-specific body part, using a computer, generating an interactive three-dimensional computer model of the identified target area based off of the acquired images of the patient-specific body part, conducting a fly through of the interactive three-dimensional computer model and identifying a treatment region of the target area, using the computer, generating a virtual reality simulation of the three-dimensional computer model and simulating a treatment plan for the treatment region, generating a physical three-dimensional object based off of the computer model after simulating the treatment plan, and practicing the treatment plan on the physical three-dimensional object.

The method also includes producing surgical phantoms based off of the physical three-dimensional object, and wherein the physical three-dimensional object is generated with densities similar to actual body parts.

In accordance with yet another preferred embodiment, the present invention provides a mixed reality simulation system of patient-specific anatomy comprising a three-dimensional visualization system that includes a non-transitory computer readable medium including computer instructions that, when executed by a processor, cause the processor to render a three-dimensional computer model of a body part based on acquired two-dimensional or three-dimensional computer images of the body part; and a 3D printer in communication with the three-dimensional visualization system, wherein the 3D printer includes a non-transitory computer readable medium including computer instructions that, when executed by a processor, cause the processor to receive the three-dimensional computer model from the three-dimensional visualization system, and print a three-dimensional physical object of the three-dimensional computer model of the body part, wherein the three-dimensional physical object includes an indicator in communication with the three-dimensional computer model. The indicator is positioned at a predetermined position about the body part or about a physical abnormality of the body part.

In accordance with another preferred embodiment, the present invention provides a mixed reality simulation system comprising: a computer operable to present an interactive three-dimensional simulation model of a patient-specific body part based on acquired two-dimensional or three-dimensional representations of the patient-specific body part; and a three-dimensional physical object that corresponds to the patient-specific body part, wherein the three-dimensional physical object includes a remote module in communication with the interactive three-dimensional simulation model.

The remote module comprises a sensor responsive to a stimulation, the sensor operable to send a signal indicative of the simulation to the computer. The remote module further comprises an actuator. The computer is operable to modify the simulation model based on the signal and operable to generate a second signal based on a received input, the input associated with a feature of the patient-specific body part. The remote module receives the second signal and the actuator generates a response based on the second signal and wherein the response is associated with a part of the object that is associated with the feature. The remote module is positioned about physical abnormalities of the body part or at a predetermined position about the body part.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a block diagram of an exemplary mixed reality simulation system in accordance with a preferred embodiment of the present invention;

FIG. 1A is a view of a CT scan of a patient-specific body part:

FIG. 1B is a perspective view of a three-dimensional computer model of the patient-specific body part illustrated in FIG. 1A and generated using the system of FIG. 1;

FIG. 1C is a perspective view of a physical three-dimensional object of the patient-specific body part illustrated in FIG. 1B;

FIG. 2 is a schematic diagram of a computer of the mixed reality simulation system of FIG. 1;

FIG. 3 is a flow chart of an exemplary method of planning and performing a medical procedure in accordance with another embodiment of the present invention;

FIG. 4 is a schematic flow chart for a method of generating a three-dimensional computer model of a patient-specific body part based on acquired images of the patient-specific body part;

FIG. 5 is a flowchart of a process of 3D imaging, visualization, and printing in pre-operative preparation and surgical training in accordance with an embodiment of the present invention;

FIG. 6 is a flowchart of a method of developing a treatment plan in accordance with an embodiment of the present invention;

FIG. 7 is a schematic view of a computer and a 3D object in accordance with an embodiment; and

FIGS. 8A-8C are views of an element of the 3D object depicted in FIG. 7.

### DETAILED DESCRIPTION OF THE INVENTION

Reference will now be made in detail to the present embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth.

Certain terminology is used in the following description for convenience only and is not limiting. The words “right,” “left,” “lower” and “upper” designate directions in the drawings to which reference is made. The words “inwardly” and “outwardly” refer to directions toward and away from, respectively, the geometric center of the identified element and designated parts thereof. Additionally, the term “a,” as used in the specification, means “at least one.” The terminology includes the words noted above, derivatives thereof and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the embodiments of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

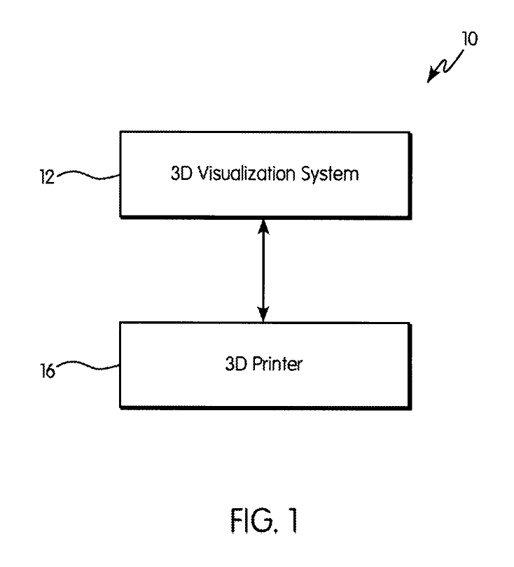

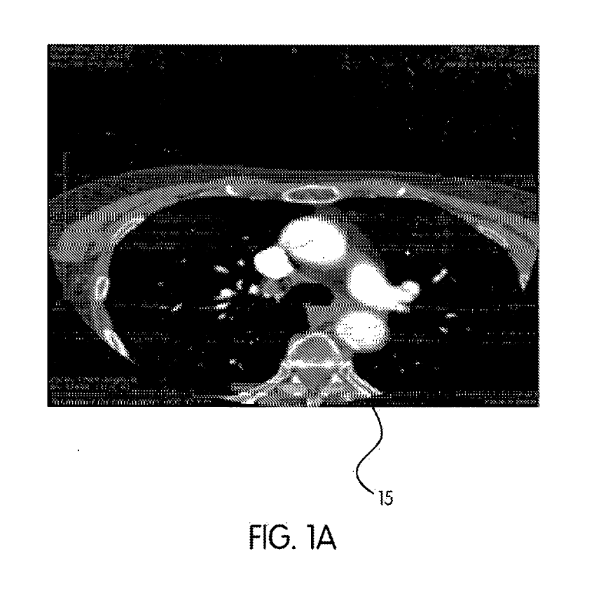

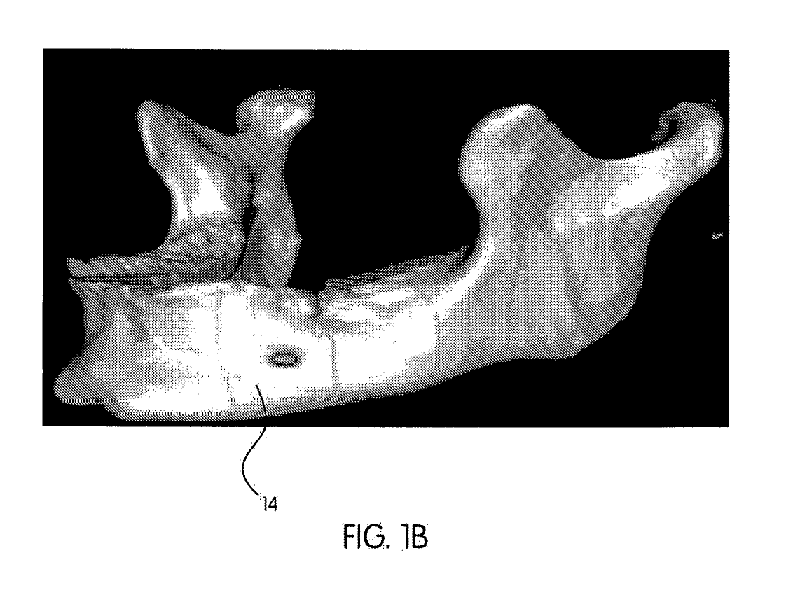

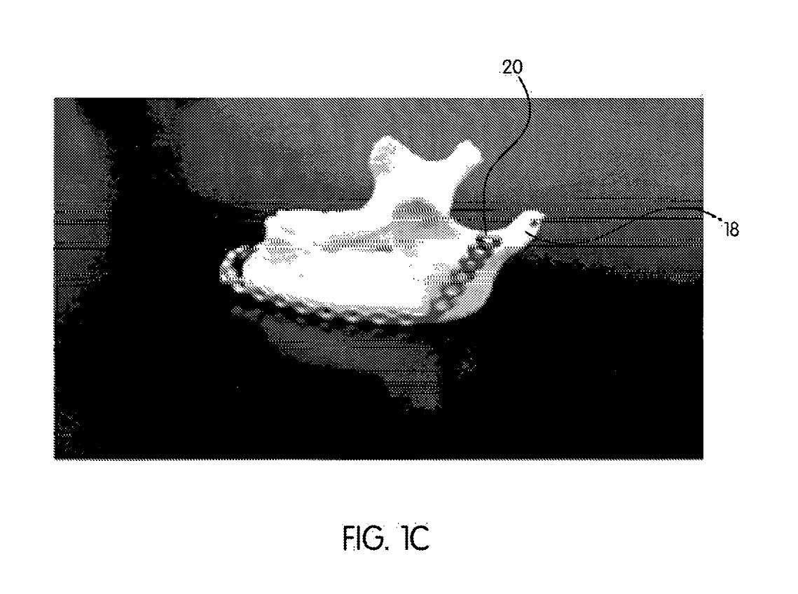

FIG. 1 illustrates an exemplary schematic diagram of a mixed reality simulation system 10 in accordance with a preferred embodiment of the present invention. The mixed reality simulation system is specifically configured to simulate patient-specific anatomy such as the mandible 15 illustrated in the CT scan figure shown in FIG. 1A. The system includes a three-dimensional visualization system 12 having a non-transitory computer readable medium including computer instructions that, when executed by a processor, cause the processor to render a three-dimensional computer model 14 (FIG. 1B) of the mandible shown based on acquired two-dimensional or three-dimensional computer images of the body part e.g., as shown in FIG. 1A.

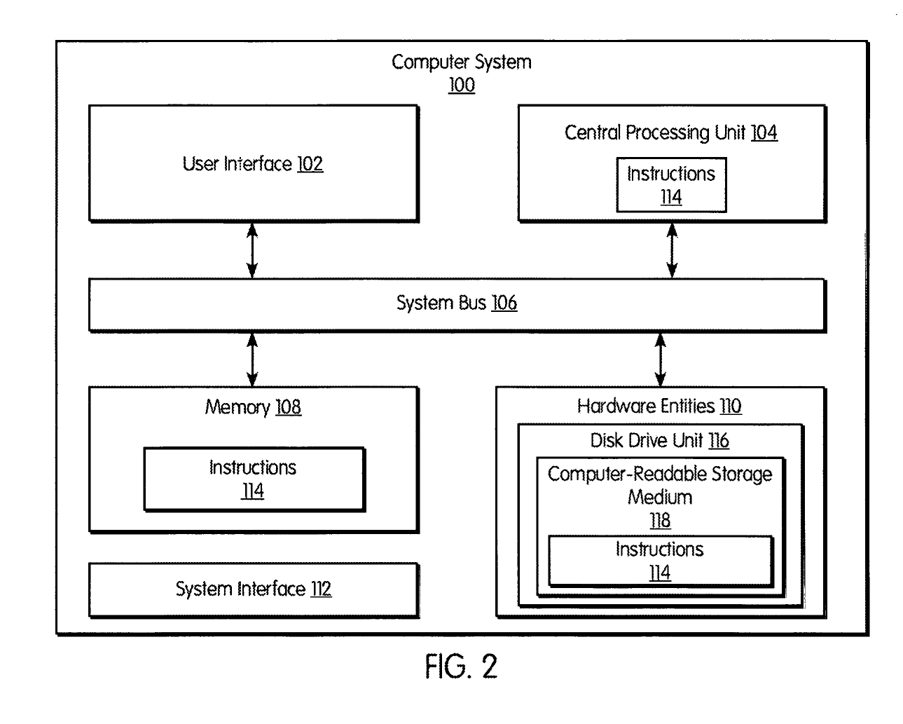

The system 10 is preferably embodied in a computer 100, as shown in FIG. 2. FIG. 2 provides a block diagram of an exemplary computer 100 implementing the present invention. In this regard, the computer 100 is generally configured to perform computer modeling in accordance with the present invention. As such, the computer 100 comprises a plurality of components 102-118. The computer can include more or less components than those illustrated in FIG. 2; however, the components shown are sufficient to disclose an illustrative embodiment implementing the present invention.

The hardware architecture of FIG. 2 represents one embodiment of a representative computing device configured to perform the invention. As such, the computer implements method embodiments of the presently disclosed invention.

As shown in FIG. 2, the computer preferably includes a system interface 112, a user interface 102, a Central Processing Unit (“CPU”) 104, a system bus 106, a memory 108 connected to and accessible by other portions of the computer 100 through system bus and hardware entities 110 connected to system bus 106. At least some of the hardware entities 110 perform actions involving access to and use of memory 108, which can be a Random Access Memory (“RAM”), a disk driver and/or a Compact Disc Read Only Memory (“CD-ROM”). System interface 112 allows the computer 100 to communicate directly or indirectly with external devices (e.g., servers and client computers).

Hardware entities 110 can include microprocessors, Application Specific Integrated Circuits (“ASICs”) and other hardware. Hardware entities 110 can also include a microprocessor programmed in accordance with the present invention

As shown in FIG. 2, the hardware entities 110 can include a disk drive unit 116 comprising a computer-readable storage medium 118 on which is stored one or more sets of instructions 114 (e.g., software code) configured to implement one or more of the methodologies, procedures, or functions described herein. The instructions 114 can also reside, completely or at least partially, within the memory 108 and/or the CPU 104 during execution thereof by the computing system 100. The components 108 and 104 also can constitute machine-readable media. The term “machine-readable media” as used here, refers to a single medium or multiple media (e.g., a centralized or distributed database, and/or associated caches and servers) that store the one or more sets of instructions 114. The term “machine-readable media,” as used here, also refers to any medium that is capable of storing, encoding or carrying a set of instructions 114 for execution by the computer 100 and that cause the computer to perform any one or more of the methodologies of the present disclosure.

Notably, the present invention can be implemented in a single computing device as shown in FIG. 2. However, the present invention is not limited in this regard. Alternatively, the present invention can be implemented in a distributed network system. For example, the present invention can take advantage of multiple CPU cores over a distributed network of computing devices in a cloud or cloud-like environment. The distributed network architecture ensures that the computing time of the statistics and enhanced functionality is reduced to a minimum, allowing end-users to perform more queries and to receive reports at a faster rate. The distributed network architecture also ensures that the implementing software is ready for being deployed on an organization's internal servers or on cloud services in order to take advantage of its scaling abilities (e.g., request more or less CPU cores dynamically as a function of the quantity of data to process or the number of parameters to evaluate).

The system 10 also includes a three-dimensional printer (also referred to herein as a “3D printer”) 16 in communication with the three-dimensional visualization system 12. The three-dimensional printer 16 includes a non-transitory computer readable medium including computer instructions that, when executed by a processor, cause the processor to acquire the three-dimensional computer model 14 from the three-dimensional visualization system 12, and print a three-dimensional physical model (also referred to herein as a three-dimensional physical object) 18 (FIG. 1C) of the three-dimensional computer model 1B of the mandible 15 shown in FIG. 1B. The three-dimensional physical model 18 includes an indicator 20 in communication with the three-dimensional computer model 14. FIG. 1C illustrates a three dimension physical object that corresponds to the mandible illustrated in FIG. 16.

The three-dimensional visualization system 12 acquires two-dimensional or three-dimensional computer images of a patient-specific body part. Two-dimensional or three-dimensional computer images can be e.g., magnetic resonance imaging (MRI), computed tomography (CT) or computerized axial tomography (CAT), positron emission tomography (PET), ultrasound, electron microscopy, or any other applicable volumetric imaging technology capable of capturing a three-dimensional object as either volumetric data or two-dimensional image data. Additionally, applicable 3D images may be captured using contact, noncontact, infrared, laser, structured light, modulated light, or passive scanning or other technology resulting in capture of a point cloud. For clarity, a point cloud is a term that corresponds, for example, to a set of data points in some coordinate system. In a Cartesian three-dimensional coordinate system, these points are usually referred to as X-, Y-, and Z-coordinates, and are sometimes used to represent a surface of a three-dimensional object (such as may be printed by a 3D printer). In certain systems, point clouds are included in output files generated by a 3D scanner when the scanner scans an object (“scanned object”). Such output files may be fed into a 3D printer in order to create a printed 3D object that corresponds to the scanned object.

Once the patient-specific computer images are acquired, the computer renders or generates the 3D computer model 14 of the patient-specific body part based on the required images. The conversion of the 2D or 3D computer image data to the 3D computer model can be accomplished by conventional software and/or algorithms readily known in the art. As such, a further detailed description of the apparatuses and method of creating such 3D computer models is not necessary for a complete understanding of the present invention. However, systems and methods applicable to the present invention include those disclosed e.g., in U.S. Pat. Nos. 5,782,762; 7,747,305; 8,786,613; U.S. Patent Application Publication No. 2013/0323700, Chapter e21 “Computer-Assisted Medical Education” Visual Computing for Medicine, Second Edition. http//dx.doi.org/10.1016/B978-0-12-415873-3.00021-3, and commercially available systems, such as, Materialise Mimics® of Leuven, Belgium; and OsiriX of osirix@osirix-viewer.com, the entire disclosures of which are incorporated by reference herein in their entirety for all purposes.

For example, the computer system 100 may be designed and programmed for navigation and visualization of multimodality and multidimensional images: 2D Viewer, 3D Viewer, 4D Viewer (3D series with temporal dimension, for example: Cardiac-CT) and 5D Viewer (3D series with temporal and functional dimensions, for example: Cardiac-PET-CT). The 3D Viewer offers all modern rendering modes: Multiplanar reconstruction (MPR), Surface Rendering, Volume Rendering and Maximum Intensity Projection (MIP). All these modes support 4D data and are able to produce image fusion between two different series (PET-CT and SPECT-CT display support). Visualization functionality may be provided through various visual interface devices such as, for example, virtual reality goggles, 3D monitors, and other devices suited for virtual reality visualization.

The 3D printer 16 can be any 3D printer capable of printing the 3D computer model 14 generated by the 3D visualization system 12. The general configuration, structure and operation of 3D printers are known in the art. As such a detailed description of its structure and operation is not necessary for a complete understanding of the present invention. However, 3D printers applicable to the present invention include those disclosed e.g., in U.S. Pat. No. 7,766,641 and commercially available systems, such as, Ultimaker² 3D Printer by Ultimaker BV of Geldermalsen, Netherlands; and LuizBot TAZ 5 3D Printer by Aleph Objects, Inc. of Loveland, Colo., U.S.A., the entire disclosures of which are incorporated by reference herein in their entirety for all purposes.

3D print refers to any of a variety of methods for producing tangible objects using additive printing that includes e.g., fused filament fabrication, stereolithography, selective laser sintering (and its variations), or electron beam melting, and the like. For example, 3D prints may be produced in different materials, using different technologies to achieve different ends. 3D printing also allows for the use of varying materials to simulate actual anatomic and physical properties.

Exemplary 3D Print from Medical Images

Medical images are typically acquired as DICOM files. DICOM stands for Digital Imaging and Communications in Medicine and is a standard for handling, storing, printing, and transmitting information in medical imaging e.g., a CT (computed tomography) scan of a patient. The DICOM file exists as a series of many hundreds of cross-sectional slices taken through an area of the patient's body via the CT scan; the combination of all of these 2D cross-sections creates a three-dimensional volume which can be processed into a file suitable for 3D printing. The process allows for separate models to be generated and/or points of interest isolated according to, for example, density e.g. soft tissue, cartilage, bone and enamel. The 3D data from the DICOM file is processed in order to extract the areas of interest.

The DICOM data can be isolated to show only the areas of interest, these can include e.g., soft tissue, bone and tooth enamel and can be recombined to print different tissue in different colors. By converting the DICOM file to the universally accepted STL format the model can be produced with any commercially available 3D printing or additive manufacturing system. It also allows the integration with other Computer Aided Design (CAD) systems and enables the development of prostheses or implants which may in turn also be produced through additive layer manufacturing techniques. By using this open format there is a lot of flexibility in the secondary processes that are available. An STL file is a triangle mesh and a universally accepted file type used in 3D printing.

In accordance with an embodiment, the present invention provides a method for interactively communicating and displaying patient-specific information. The method includes acquiring two-dimensional or three-dimensional computer images of a patient-specific body part. Then, using a computer e.g., the 3D visualization system 12, generating an interactive three-dimensional computer model based off of the acquired computer images of the patient-specific body part. Thereafter, a physical three-dimensional model 18 based off of the computer model is generated.

Preferably, the three-dimensional model 18 is generated by 3D printing the computer model. While generating the 3D physical object, the physical object is incorporated with one or more indicators 20. Indicators can be e.g., a visual indicator, an audio indicator, a vibratory indicator, or the like. In certain embodiments a radioactive indicator may be used for targeting of radiation therapy . . . . For example, the visual indicator can be a light emitting diode (LED) indicator, while the audio indicator can be an audio alarm. An indicator may also be a device that can generate a vibration or cause movement of a part of the physical object such as an actuator. The indicator 20 is configured to be in communication with the computer. Such communication may take place through a wire connection or through a wireless connection such as WiFi, Bluetooth, or some other radio-based wireless communication standard. Thus, when a user operates the computer and interacts with the 3D computer model, the computer may communicate with the indicator in the physical object 18 in order to produce a perceptible interaction with the physical object via the indicator.

In certain embodiments, such as embodiments wherein radiation treatments are planned the physical object may have a radiation sensor implanted to provide feedback to the health care professionals planning a radiation procedure regarding the amount and directionality (power and angle) of radiation applied to a targeted region of a body part. In such embodiments the radiation sensor may be, for example, an implantable MOSFET radiation dosimeter such as those manufactured by Sicel Technologies (Morrisville, N.C., USA.)

In certain embodiments, the physical body part may be a surgical phantom. Surgical phantoms refer to body parts that are built to simulate the look and feel of human anatomy. Surgical phantoms may be created through various means including 3D printing, fabricating components and painting components, or using various materials to simulate the density of organs, tumors, and the like. In certain examples, 3D printing of an organ such as a spleen may be performed by printing the outer surface of the organ in a very thin, pliable material. Once the outer surface is printed it may be filled with a material such as a low density gel. Such a filling allows the creation of a physical object that can simulate the density and behavior of an actual spleen. Physical objects fabricated according to such a method allow radiation targeting and other surgical simulations to be more accurate.

An example of a simulation utilizing a phantom may, among other things, include the printing of a physical object that corresponds to a patient-specific cancerous organ, where the cancerous organ has within it a cancer tumor. Such a physical object may be created based on images of a patent-specific body part (the organ and the tumor) through techniques disclosed herein. The physical object itself may be created through various techniques, for example it may be 3D printed or may be created through sculpture or molding that is done using other materials. Such a physical object may be created as a number pieces of pieces that can be assembled together to form the complete physical object that corresponds to the patient-specific cancerous organ. In an example, where the physical object corresponds to an organ that contains a tumor, the physical object may be assembled from a number of pieces that, when assembled have a cavity inside. Such a cavity may be filled with a physical object that corresponds to the shape and size of the tumor. Thus, physical object can be assembled as a small (tumor-sized) physical object embedded within a larger (organ-sized) physical object that corresponds to the organ. Such an object can be useful in the preparation for a procedure that is targeted at the tumor in the patient's organ, as the physical object that corresponds to the tumor occupies the analogous position and size of the actual tumor being targeted. Of course such a physical object (or set of physical objects) may be instrumented as disclosed herein in order to improve the accuracy and sensitivity of procedures performed on the test case of the physical object.

In a procedure performed on a physical object shaped in that manner, the physical object may be treated as a “surgical phantom” of the organ with the tumor. The surgical phantom may be sliced, irradiated or otherwise interacted with to simulate the procedure. Naturally, such procedures on the surgical phantom may be performed and re-performed on one or more surgical phantoms as preparation for the procedure. For example, such a procedure may be utilized to modify and select the size and angle of a radiation beam being applied to the surgical phantom to ensure that there is complete immersion of the tumor while minimizing the beam's impact on healthy cells when the procedure is applied to the patient. In a specific such example, for a procedure with a radiation beam (or scalpel procedure) that is anticipated to be 5 mm wide, the slicing (or irradiation) of the phantom will be in the same widths and angles as would be used on the patient. The phantom may then be evaluated to determine the impact on the cancerous and healthy cells to maximize destruction of the cancerous cells and minimize injury to the healthy cells.

Other applications utilizing phantoms may be developed in the future that are not envisioned here, but would incorporate the products developed here.

Each of the one of more indicators 20 can be positioned at or about predetermined positions of the body part. For example, the indicator 20 can be positioned at a fixed location, such as, a tumor site of the body part, or a plurality of indicators can be arranged in a two-dimensional or three-dimensional array. Each indicator can also be configured to be in communication with the computer via a transmitter/receiver and the like. Additionally, the indicator is preferably positioned about physical abnormalities of the patient-specific body part.

For example, when a user executes a fly-through of the 3D computer model 14 and tags e.g., an anatomical anomaly, the corresponding anatomical anomaly generated in the 3D physical model is indicated via the indicator 20. As used herein, “fly-through” refers to a user viewing a computer model as if they were inside it and moving through it, as well as a computer-animated simulation of what would be seen by one flying through a particular region of the computer model.

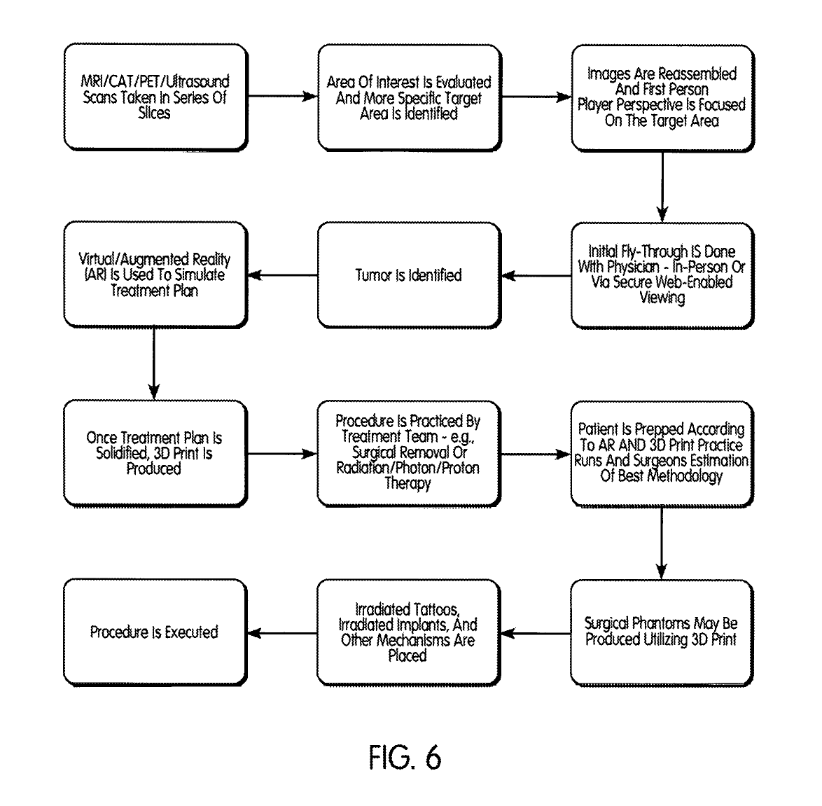

In accordance with another embodiment, the present invention provides a method of developing a medical treatment plan. Referring to FIG. 6, the method includes acquiring two-dimensional or three-dimensional computer images of a patient-specific body part, and identifying a target area of the patient-specific body part. The method also includes using a computer to generate an interactive three-dimensional computer model of the identified target area based off of the acquired images of the patient-specific body part, conducting a fly-through of the interactive three-dimensional computer model and identifying a treatment region of the target area, generating a virtual reality simulation of the three-dimensional computer model, and simulating a treatment plan for the treatment region. The method also includes generating a physical three-dimensional object based off of the computer model after simulating the treatment plan, and practicing the treatment plan on the physical three-dimensional object.

Virtual Reality Simulation

In accordance with another embodiment, the present invention provides for 3D virtual reality simulation (i.e., 3D visualization) of the 3D computer model 14. 3D visualization generally refers to any of a series of software driven algorithms of re-assembling the 2D images that result from a point cloud, MRI/CAT or other scan into a composite image that can be represented to simulate three dimensions for presentment and manipulation on a two-dimensional screen or presented to the viewer through specialized glasses e.g., Oculus Rift™ and Google Goggles™ or converted into a full three-dimensional image presented on a monitor intended for that purpose, similar to the difference between watching a movie in 2D or 3D (complete with glasses). 3D computer models can be converted to 3D visualization using software such as Mimics™, Osirix™, a variety of video gaming software technology, 3D image rendering software, and other related technologies readily known in the art. As such a detailed description of them is not necessary for a complete understanding of the present invention.

Another aspect of the virtual reality simulation system may include Augmented Reality (AR). AR superimposes a computer-generated image on a user's view of the real world, thus providing a composite view. Such functionality may be provided to a user of the virtual reality simulation system by, for example, outfitting a user with specialized equipment (for example, instrumented gloves, instrumented glasses, instrumented head phones) that allow the user to interact directly with reality while also providing additional stimulation (e.g., a visual overlay, tactile feedback, audible signals) that may be experienced simultaneously with the direct interactions with reality.

The foregoing method of the present invention can be applied, for example, to radiation therapy. Among the goals of radiation therapy are to shrink tumors and kill cancer cells. While the therapy will also likely injure healthy cells, the damage is not permanent. Normal, noncancerous cells have the ability to recover from radiation therapy. To minimize the effect radiation has on the body, the radiation is targeted only to a specific point(s) in a patient's body.

Prior to radiation therapy a full simulation using the mixed reality simulation system 10 may be performed, including having the patient positioned and laser-guiding tattoos applied. A CT scan (or other similar imaging process) of the region to be treated can be done. Information from the CT scan is used to precisely locate the treatment fields and create a “map” for the physician. The physician may use such a “map” to design the treatment to fit a patient-specific case. 3D visualization and patient-specific prints can be used to minimize the risk to healthy cells and to ensure that all cancer cells are fully targeted. Based on the captured CT scan (or other similar imaging process) 3D visualization can help inform the targeting process to achieve the maximal results with minimal damage to healthy cells. The process may include pre-treatment production of 3D visualizations and/or prints, and may also include the incorporation of 3D visualization, and printing in real-time, before, during and after therapy application.

Other applications of these techniques include generating 3D visualization, and printing may also be incorporated into other medical procedures or other therapy or surgical procedure requiring targeting or removal of specific cells located within other cells that are to remain unharmed. Such processes may be completed before the procedure or delivered in just-in-time or real-time before, during or after a similar procedure.

Exemplary Method for Strategic Medical Procedure Implementation

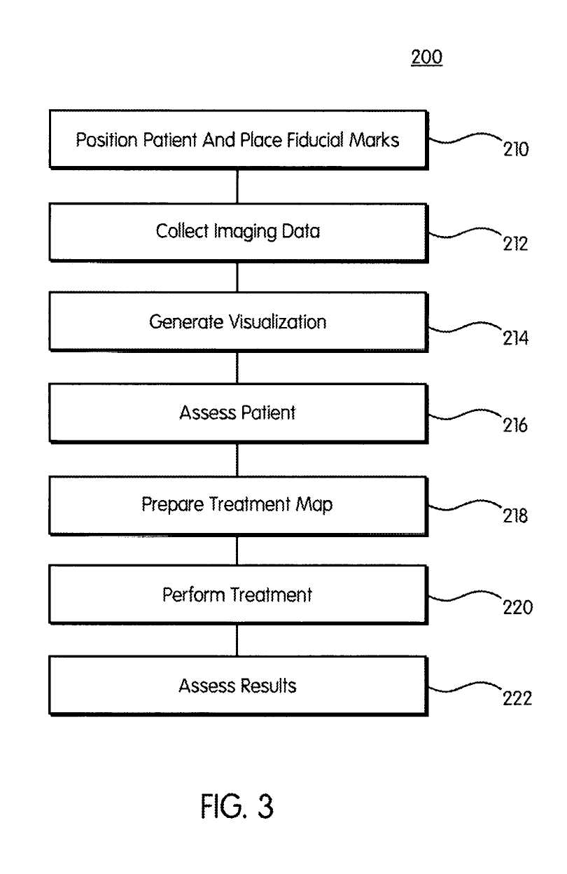

FIG. 3 is a flow chart showing an example process 200 for performing a medical procedure in accordance with an embodiment of the system 10 and method as disclosed herein. In some implementations, some of all of the process steps 210-222 may be performed by the system 10. A particular order and number of steps are described for the process 200. However, it will be appreciated that the number, order, and type of steps required for the process 200 may be different in other examples.

In step 210, a patient is positioned and fiducial marks are placed to assist in the process of performing a scan of the patient. The patient is positioned and the marks are placed in order to optimize the process of scanning. Optimized scanning generates accurate and thorough imaging for all body parts subject to the procedure. Fiducial marks are indications made to guide the scanning process. In certain embodiments a laser-guidance tattoo is used as a fiduciary mark. In step 212 image data is collected. Image data may be collected through one or a combination of methods. Examples of methods include: CT scanning, MRI, X-Ray, or other methods suitable to generate a 3D image of a body part.

In step 214, visualization is generated based on the results of the scan. The visualization may be presented, for example, on a display such as the user interface 102. In certain embodiments the visualization may be presented so as to allow a user to experience fly-through of the visualization. Such embodiments may include virtual reality elements or allow a user to experience the visualization as a virtual reality experience.

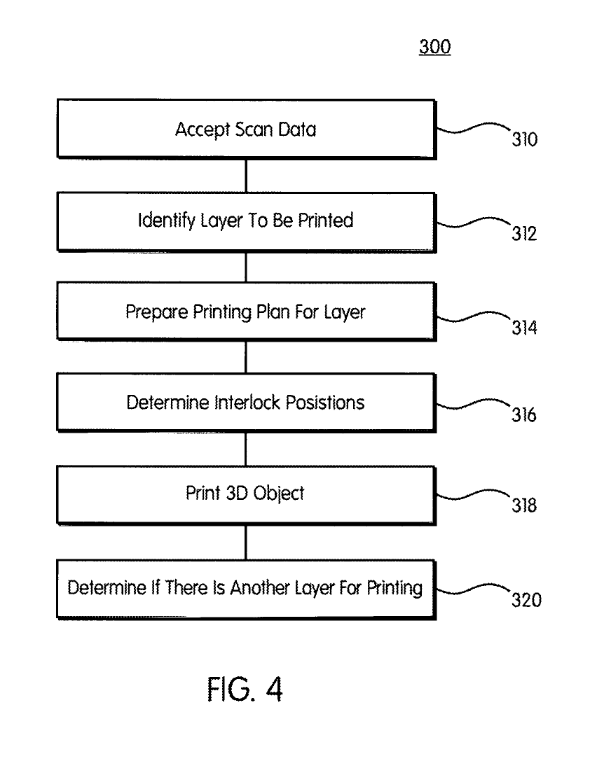

In certain embodiments the visualization may include (in addition to or instead of the visualization presented on the user interface 102) an object such as may be generated by a 3D printer. FIG. 4 is a flow chart showing steps that may be used in certain embodiments to generate an object as part of a visualization as generated according to step 214.

Referring to FIG. 4, in step 310, scanning data is accepted for analysis on a processor such as Central Processing Unit 104. In step 312 a layer to be printed is identified. In certain embodiments the scan data may be used to generate a series of layers that together make up the section of the body that is the target of the procedure and the sections of the body around it. In embodiments in which multiple layers are to be printed the 3D printing process selects a particular layer for each printing “run” of the 3D printer. A run of the 3D printer creates a single piece which may be all of a layer or a portion of a layer. The determination of whether a piece is a complete layer, that is, if a layer is printed in a single run or whether the layer is printed as a series of pieces is made in step 314 in which a printing plan is prepared.

In step 316, based on the printing plan a determination of interlock positions is made. The interlock positions are points on a piece that connect with other pieces to make a connected layer or points on a particular layer that connect with other (e.g., neighboring) layers. The positioning of the interlocks is made in order to allow each layer and the layers around it to have structural integrity. The interlock positions are also selected so that they minimize the interference with the visualization process used by the professionals planning the procedure.

In step 318, a 3D object is printed. The 3D object may be printed as a single piece, as a series of pieces each of which corresponds to one of several layers (that are connected at interlock points) or as a series of pieces that make up a layer which connects to other layers. In step 320, a determination is made as to whether there are other layers to be printed.

Referring back to FIG. 3, in step 216, the patient is assessed based on the scan data and the visualization. In step 218, a treatment map is prepared based on the patient assessment made in step 216. The treatment map, in certain embodiments may involve a selection of a certain region for more detailed scanning, a certain region to be targeted for radiation or chemotherapeutic treatment, or another medical procedure that will perform an action on a part of a patient's body as determined by the visualization and the assessment of the patient. In step 220 the patient is treated. In step 222, the results of the treatment are assessed. Based on the assessment performed in step 216 the process described in flow chart 200 may be repeated in order to improve the result or patient outcome.

With regard to the flow charts described above, implementing software is designed to guide practitioners in generating strategic plans for medical procedures, including target area selection and planning. Medical procedure activities may be guided by the implementing software's Graphical User Interface (“GUI”) for practitioner users to easily plan and implement their efforts in valid and reliable ways to permit meaningful outcome evaluations. With regard to steps mentioned above, repeated iterations of the implementing software, subsequently informed intervention activities, and outcome (re)evaluations can be used to heuristically assess the success of the planned procedure.

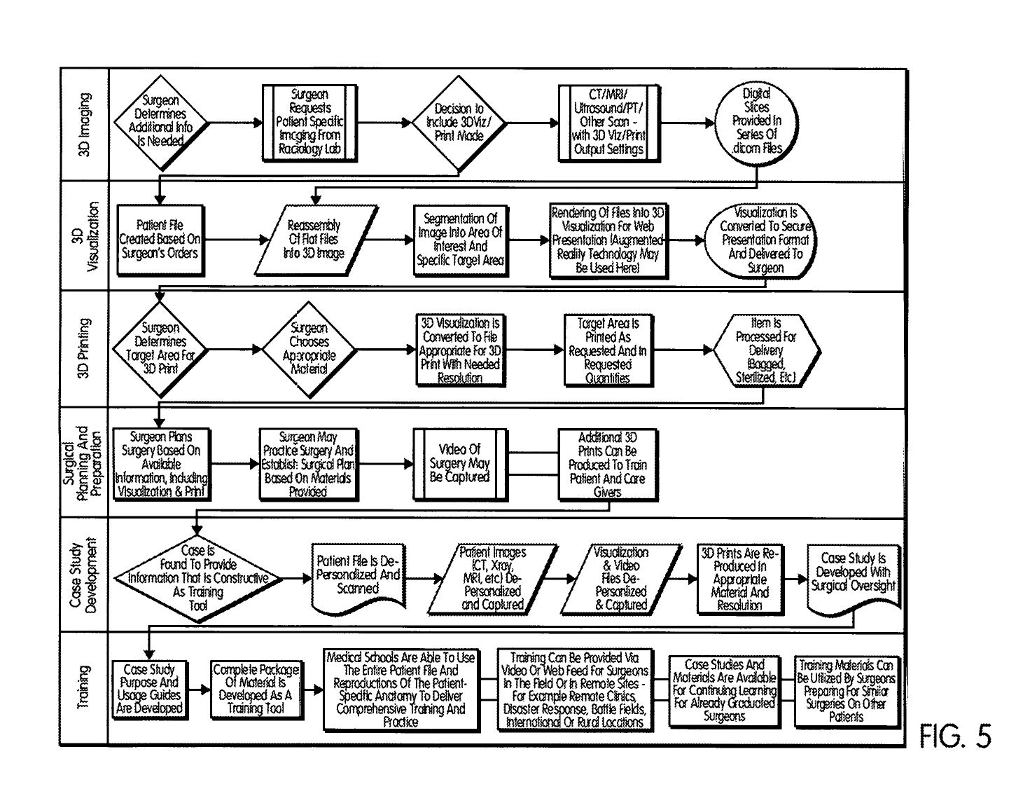

In accordance with another embodiment, the present invention provides a system and method of 3D imaging/visualization and printing in pre-operative preparation and surgical training, as illustrated in FIG. 5. Referring to FIG. 5 the method includes the steps of 3D imaging, 3D visualization, 3D printing, surgical planning and preparation, case study development and training. The method can be used to produce support tools or medical training tools for training and educating users e.g., doctors, surgeons, and students, on various procedures.

3D Imaging Process

The user (e.g., doctor or surgeon) determines that more information is necessary to identify a treatment plan for a particular patient. Additional information may include X-rays, MRI, CT Scans, etc.

The process typically starts in the radiology lab as a part of the normal process of developing the information package that will be delivered to the doctor/surgeon. Making the decision at this point will allow the technicians to capture the images in the optimal way to deliver all the information requested.

In more complex or novel situations, the information provided by the standard protocol of lab tests and images are not sufficient for a surgeon to be fully prepared for operation. In these cases, additional information through 3D visualization and 3D printing is utilized. The decision to utilize the mixed reality simulation system is made as early in the process as possible.

The radiology technician executes the order for imaging, taking into account the demand for 3D visualization and 3D print outputs. For example, MRI scans can be captured as slices of thicknesses as small as 0.5 mm to as large as 5.0 mm. The resolution is determined both by the settings on the imaging machine (the better the resolution, the longer the scan takes) and by the processing that the computer of the mixed reality simulation system runs after the patient has left the MRI machine. 3D visualization is more accurate the higher the resolution. 3D printed objects can achieve resolutions as fine as 16 μm (0.016 mm), making the limiting factor the quality of the image captured.

3D Visualization

The mixed reality simulation system creates an electronic patient file based on the doctors/surgeon's direction, incorporating the output options made and preparing the system for incorporation of DICOM files that are generated by the radiology lab. DICOM files are the typical file format produced by imaging software systems. The DICOM files are reassembled and converted into a 3D composite image.

The 3D image is segmented to focus on the area of interest and the specific target area. For example, in preparation for repairing an aortic dissection, a surgeon may request that the visualization focus on the aorta itself as the area of interest and identify the likely target area as being the 10 mm area closest to the heart.

The system converts the area of interest into a 3D visualization that allows the surgeon to do a virtual fly-through of the area, including additional magnification options along the area of interest and specifically on the target area.

Augmented Reality technology may be integrated to give the surgeon a greater ability to visualize the area of interest and plan the treatment/surgery accordingly. For example a surgeon may don a wearable computing device (e.g., google glass) that allows the surgeon to view and handle a physical object (e.g., a 3D printed object that corresponds to a body part, or even an actual body part on a patient) and to receive, in addition to the tactile and visual experience of the physical object, information from the computer that corresponds to aspects of the physical object and augments the experience of the physical object.

The 3D visualization can be presented via secure web channel directly to the surgeon. A video capture of the fly-through can also be produced to be shared with the rest of the surgical team and possibly used as a tool for explaining the procedure to the patient and family. The fly-through informs the decisions that must be made for the surgical intervention, including e.g., how to position an implant, what size device to prepare, what tools need to be ready, etc.

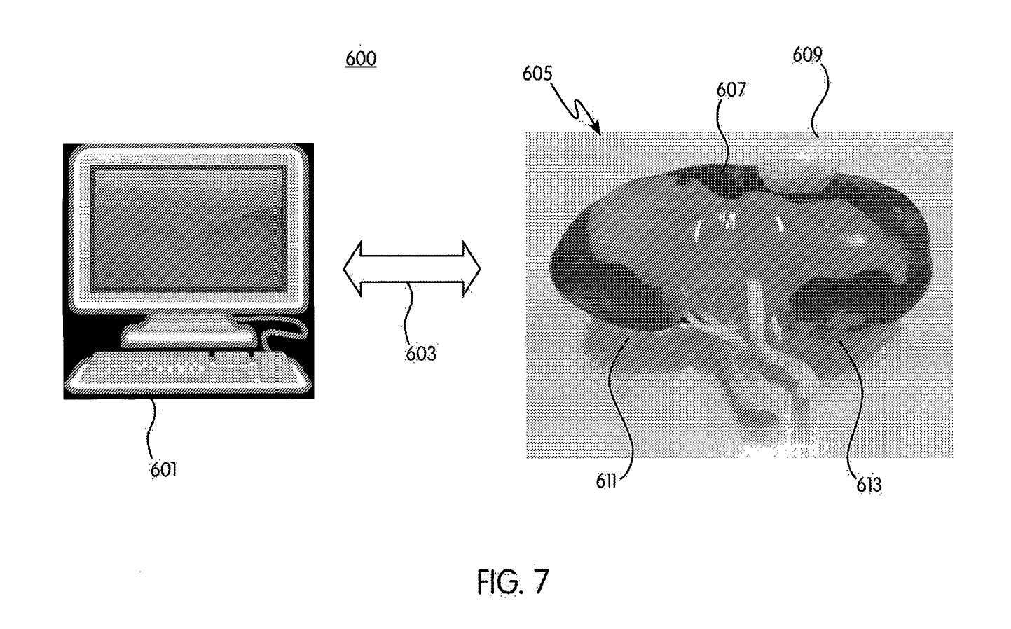

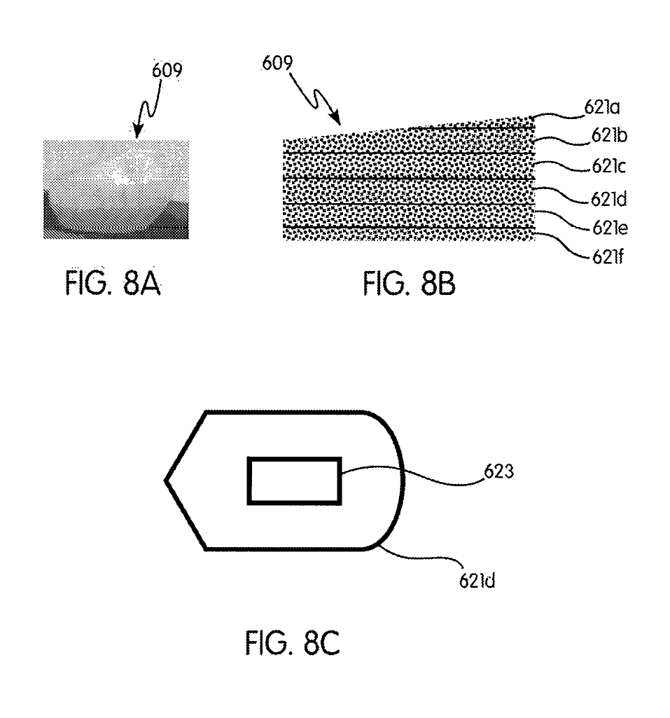

A system 600 in accordance with an embodiment of the invention is illustrated in FIGS. 7 and 8A-8C. Computer 601 communicates with physical object 605 via a communication channel 603. Communication channel 603 may be any of various wireless communication protocols suitable for transferring commands to/from an indicator placed inside of or in physical proximity to the physical object. The indicator may, for example, be a small computing device which schematically corresponds to the computer illustrated in FIG. 2. In addition to the computing and communication components the indicator may include other components such as a sensor and/or a feedback device. Such sensors can detect pressure, the presence of a pointer such as a magnet, light, or other types of interactions with the physical object. Feedback devices that be integrated into the indicator include devices that generate outputs that are human perceptible for the physicians and patients who interact with the physical object. Such outputs may be tactile (i.e., vibration), audible (e.g., sounds or alerts that may be given), visual (e.g., LEDs), or other suitable outputs.

Such indicators may be based on, for example, smart sensors such as a waspmote sensor device available from Libelium (www.libelium.com/products/waspmote/).

FIG. 7 also shows a physical object 605. The physical object corresponds to a patient-specific body part that is the subject of a medical procedure. The physical object may be made up of a several different components 607, 609, 611 and 613. In certain embodiments of the present disclosure each of the components are printed together so that the entire physical object is printed as a whole in one pass of the printer. In other embodiments each of several components of the physical object may be printed separately and assembled in order to form the full physical object. Such assembly may be performed by including interlocking pieces in the printing process or by simply gluing or otherwise attaching the pieces together to form the full physical object.

Note that any or all of the components may be printed of distinct materials. The properties of the material used in printing the components may be selected to support the expected interaction with the doctor/surgeon and patient with the system. For example, if the physical object is instrumented with an indicator that provides a visual signal from within the physical object based on an interaction (with either the physical object or with the virtual model) the material used in printing the affected component should be transparent or translucent.

FIG. 8A shows a particular component 609 of physical object 605. FIG. 8B is a schematic side-view of component 609 that illustrates an embodiment wherein the component has been printed as a series of slices (621a-621f) which when assembled form the full component 605.

In certain embodiments wherein a component (or a set of components) are printed in slices, a 3D splitter program (such as, 3D Splitter from aescripts, www.aescripts.com) may be used to generate the slices to be printed. For example, the 3D splitter first receives an electronic file that corresponds to the component to be printed. The 3D splitter program then processes the electronic file and generates a set of other files (“sub-files”) each of which can be printed individually by a 3D printer. When each of the sub-files is printed, a user assembles the outputs (each associated with a particular sub-file) so that the set of outputs can be assembled in order to form the physical object that was described in the original electronic file.

Among the advantages of processing an electronic into a set of sub-files is the ability to easily generate one or more cavities or channels in the fully assembled physical object. Such a cavity is illustrated in FIG. 8C which shows a particular slice 621d of physical object 605 which has been printed with a rectangular hole 623 within the boundaries of the printed slice. Such a hole is suitable to receive an indicator that can be used to communicate wirelessly to/from the physical object (or component of the physical object) to the computer 601. It should be noted that in a similar manner as the hole may be printed in a particular slice, a channel may be created through the printing of a particular slice of the physical object. Such a channel may be suitable for running wires that carry either or both of signals or power to the indicator inside of the component of the physical object.

Note that in certain embodiments a selection of a particular component or aspect of a patient body part may be selected as one for which interactive responsiveness should be provided. Such a selection will be used by the computer to determine which component (and/or slices of components) should have a cavity or other accommodation suitable for accepting an indicator. The indicator (when it receives a signal from the computer on which the 3D virtual model is presented) can then provide the interactive responsiveness necessary.

3D Printing

The surgeon identifies the specific target area for 3D printing. This may be, in the example of the aorta, the segment closest to the heart. In the case of a scoliotic spine, the print may be of the entire spine so that the surgeon can accurately plan the size and type of the pedicle screws and rods that would be needed.

Surgeon chooses appropriate material—for example, FFF (that is, “fused filament fabrication”) for use fabrication of the object for use in explanation to the patient, SLA (that is, stereolithography) in order to generate an object fabricated in an autoclavable material (that is, a material that can be processed by an autoclave while maintaining its structural integrity). The object fabricated in the autoclavable material is appropriate for use in the operating room and as a high-resolution guide for pre-operative planning. Or perhaps a composite material that simulates bone for performing practice cuts in advance of a complicated procedure.

The system then converts the file produced for the visualization into a file ready for 3D print, at the appropriate resolution and utilizing the correct combination of technology and materials. The target area is printed as requested and in the requested quantities and materials. The necessary post-processing is performed, including cleaning support material, sterilization, bagging, etc.

Planning and Preparation

Surgeon plans the surgery based on the available information, including MRIs, CTs, 3D visualization, 3D prints, etc. The surgeon may practice the surgery and establish the surgical plan based on the information package provided from all sources. In certain embodiments, such as embodiments that support a preparation for a radiation treatment, the Virtual or Augmented Reality system may be utilized to simulate a body part undergoing treatment from different positions or orientations. Such embodiments allow simulations of radiation therapies from a number of radiation targeting devices. Simulations using such embodiments can be used to orient the radiation targeting device(s) so that the radiation can be applied in the optimal degree and dosage for a tumor in the effected body part being targeted.

The surgery itself can be captured on video along with the information provided by the surgical team during the surgery as part of the audio of the surgery (audio may be added or edited in after the surgery). Additional 3D prints can be produced to train the patient and care givers based on the surgery as it happened and any post-operative imaging.

Case Study Development

Using the integrated virtual reality and physical object system as generated in accordance with an embodiment of the system disclosed herein, a patient's case may be evaluated considering all of the information available and the patient's and surgeon's acquiescence for use as a training tool. Once agreed, the patient file is depersonalized and the relevant information is digitally captured for inclusion. No editing is done on the files except to remove extraneous materials, at the surgeon's discretion. Patient images and lab results are also depersonalized and captured digitally. The visualization and video files are also depersonalized and captured.

3D prints are produced in the appropriate material, process, and resolution. In some cases multiple products may be produced individually or in bulk quantities. For example, for general medical training, a lower-resolution, lower-cost product may be used. For surgeons using the case study to learn and practice the surgery, the models produced will be higher-resolution and will utilize available materials that are best suited for use in practice.

The final case study is developed with surgical oversight with a focus on capturing the full experience of identifying symptoms, a diagnosis methodology, treatment plan, the tools used to develop and implement the treatment plan, the surgery, the results and the post-operative assessment.

Training

The case study purpose and usage guides are developed. This includes a basic lesson plan for the most effective utilization of the case study and materials. The complete package of materials is developed as a training tool. Instructors are able to order additional 3D printed materials as needed for their particular student population.

Medical schools are able to use the entire patient file and reproductions of the patient-specific anatomy to deliver comprehensive training and practice. This frees the school from the constraints of a limited and expensive cadaver pool and the fact that cadavers can only present the anatomy they have. It is not possible to acquire a cadaver with a specific set of anomalies to complement the classroom teaching going on at the time. In the same way, training of surgical residents is also captive to the clinical realities of the day and cannot be readily targeted to develop a residents training in a logical sequence.

In sum, the present system and method is provided for by a mixed reality simulation system that includes a three-dimensional computer model of a patient-specific body part based on acquired two-dimensional or three-dimensional computer images of the body part, and a three-dimensional physical object of the patient-specific body part based on the three-dimensional computer model, wherein the three-dimensional physical object includes an indicator in communication with the three-dimensional computer model.

Surgical preparation and training can be provided via video or web feed for surgeons in the field or in remote sites—for example remote clinics, disaster response, battle fields, international or rural locations. The ability to provide support and training, where needed and when needed is an incredible advancement for medical provision worldwide.

Case studies and materials are available for continuing learning for already graduated surgeons. For the surgeon preparing to treat a patient with a rare or complex illness, the ability to prepare fully by utilizing these case studies along with the opportunity to purchase 3D visualization and 3D printed materials for his/her specific patient will make an incredible impact on the medical field in the U.S. and around the world.

Training materials can be utilized by surgeons preparing for similar surgeries on other patients.

Patient-specific training materials can also be utilized to train the patient and his or her care-givers in the particular needs of the patient. For example, the 3D print, 3D visualization and related materials can be utilized to give the parents of the child with a severely scoliated spine instruction in how to hold the child properly, even how to place the child in a car seat or other device to best protect the child in various positions.

Applications

The current application discussed above focuses on the use of the mixed reality system and method in the surgical pre-operative planning, preparation, and medical training spheres. However, the present mixed reality simulation system may also be utilized in, for example, molecular biology, dentistry, veterinary medicine, astronomy, archeology, anthropology, and others areas where there is a benefit to converting two-dimensional images into composite three-dimensional visualizations and objects for the purposes of better understanding, assessing, studying and teaching about a particularly complex concept.

It will be appreciated by those skilled in the art that changes could be made to the embodiments described above without departing from the broad inventive concept thereof. For example, additional components and steps can be added to the various systems and methods disclosed. It is to be understood, therefore, that this invention is not limited to the particular embodiments disclosed, but it is intended to cover modifications within the spirit and scope of the present invention as defined by the appended claims.

1. A method for interactively communicating and displaying patient-specific information comprising:
acquiring two-dimensional or three-dimensional images of a patient-specific body part;
using a computer, generating an interactive three-dimensional computer model based off of the acquired images of the patient-specific body part;
generating a physical three-dimensional object based off of the computer model;
incorporating one or more indicators into the physical three-dimensional object that are each in communication with the computer;
interacting with the computer model to select an attribute of the body part; and
indicating on the physical three-dimensional object the selected attribute with the indicators.

2. The method of claim 1, further comprising a step of receiving a selection of a feature of the patient-specific body part for providing interactive responsiveness.

3. The method of claim 2, wherein the step of generating a physical three-dimensional object comprises printing a plurality of pieces that assemble to form the physical three-dimensional object and the assembled physical three-dimensional object defines a cavity suitable to receive the indicator.

4. The method of claim 3, furthering comprising the step of positioning the indicator in the cavity, the indicator operable to respond to a signal from the computer and provide a physically tangible response associated with the three-dimensional object.

5. The method of claim 1, wherein the step of incorporating the indicators into the physical three-dimensional object is conducted during the step of generating the physical three-dimensional computer model.

6. The method of claim 1, wherein the indicators are visual, audio or vibratory indicators.

7. The method of claim 1, wherein the step of interacting with the computer model, comprises conducting a fly-through of the interactive three-dimensional computer model.

8. The method of claim 1, wherein the step of generating the physical three-dimensional object includes printing the physical three-dimensional object.

9. A method of developing a medical treatment plan comprising:
acquiring two-dimensional or three-dimensional computer images of a patient-specific body part;
identifying a target area of the patient-specific body part;
using a computer, generating an interactive three-dimensional computer model of the identified target area based off of the acquired images of the patient-specific body part;
conducting a fly-through of the interactive three-dimensional computer model and identifying a treatment region of the target area;
using the computer, generating a virtual reality simulation of the three-dimensional computer model and simulating a treatment plan for the treatment region;
generating a physical three-dimensional object based off of the computer model after simulating the treatment plan; and
practicing the treatment plan on the physical three-dimensional object.

10. The method of claim 9, further comprising producing surgical phantoms based off of the physical three-dimensional object.

11. The method of claim 9, wherein the physical three-dimensional object is generated with densities similar to actual body parts.

12. A mixed-reality simulation system of patient-specific anatomy comprising:
a three-dimensional visualization system that includes a non-transitory computer readable medium including computer instructions that, when executed by a processor, cause the processor to
render a three-dimensional computer model of a body part based on acquired two-dimensional or three-dimensional computer images of the body part; and
a 3D printer in communication with the three-dimensional visualization system, wherein the 3D printer includes a non-transitory computer readable medium including computer instructions that, when executed by a processor, cause the processor to
receive the three-dimensicnal computer model from the three-dimensional visualization system, and
print a three-dimensional physical object of the three-dimensional computer model of the body part, wherein the three-dimensional physical object includes an indicator in communication with the three-dimensional computer model,
wherein the three-dimensional physical object includes a remote module, the remote module including an actuator in communication with the three-dimensional visualization system and a sensor responsive to a stimulation, the sensor operable to send a signal indicative of the stimulation to the three-dimensional visualization system.

13. The system of claim 12, wherein the indicator is positioned at a predetermined position about the body part.

14. The system of claim 12, wherein the indicator is positioned about a physical abnormality of the body part.

15. A mixed reality simulation system comprising:
a computer operable to present an interactive three-dimensional simulation model of a patient-specific body part based on acquired two-dimensional or three-dimensional representations of the patient-specific body part; and
a three-dimensional physical object that corresponds to the patient-specific body part, wherein the three-dimensional physical object includes a remote module in communication with the interactive three-dimensional simulation model,
wherein the remote module further comprises an actuator.

16. The system of claim 15 wherein the remote module comprises a sensor responsive to a stimulation, the sensor operable to send a signal indicative of the simulation to the computer.

17. (canceled)

18. The system of claim 16, wherein the computer is operable to modify the simulation model based on the signal.

19. The system of claim 18, wherein the computer is operable to generate a second signal based on a received input, the input associated with a feature of the patient-specific body part.

20. The system of claim 19, wherein the remote module receives the second signal and the actuator generates a response based on the second signal and wherein the response is associated with a part of the object that is associated with the feature.

21. The system of claim 15, wherein the remote module is positioned about physical abnormalities of the body part.

22. The system of claim 15, wherein the remote module is positioned at a predetermined position about the body part.
